(19) United States
(12) Patent Application Publication
(10) Pub. No.: US 2025/0064051 A1
(43) Pub. Date: Feb. 27, 2025

(54) NORMOTHERMIC PERFUSION OF KIDNEYS

(71) Applicant: Saint Louis University, St. Louis, MO (US)

(72) Inventors: Mustafa Nazzal, Ellisville, MO (US); Ajay Jain, St. Louis, MO (US); Yasar Caliskan, St. Louis, MO (US); Kento Kurashima, St. Louis, MO (US)

(73) Assignee: Saint Louis University, St. Louis, MO (US)

(21) Appl. No.: 18/813,910

(22) Filed: Aug. 23, 2024

Related U.S. Application Data
(60) Provisional application No. 63/578,720, filed on Aug. 25, 2023.

(51) Int. Cl. A01N 1/02; G01N 33/50

(52) U.S. Cl. CPC A01N 1/0247; G01N 33/5088

(57) ABSTRACT

Methods and systems provide a human kidney perfusion model with dual blood supply channels, permitting two kidneys from the same donor to be pumped simultaneously for rigorously controlled therapeutic testing.

### CROSS REFERENCE TO RELATED APPLICATIONS

This application claims the benefit of U.S. Provisional Application No. 63/578,720, filed Aug. 25, 2023, the contents of which are incorporated by reference herein in their entirety.

### FIELD OF THE INVENTION

The present invention relates to methods and systems that provide for a human kidney perfusion model with dual perfusate supply channels, permitting two kidneys to be pumped simultaneously.

### BACKGROUND OF THE INVENTION

The organ supply shortage is a major challenge facing the field of organ transplantation. Transplant nephrology has faced an increasing demand for innovative practices to optimize kidney graft success. The high rate of discard in kidney transplantation has created discussion on improving transplant outcomes of kidneys from expanded criteria donors (ECD) and donation after circulatory death (DCD). Growing research suggests that expanding the criteria for optimal kidney donation increases survival rates amongst waiting kidney recipient candidates although this presents an increased risk for graft loss. At a lower estimated longevity rate and a greater risk of warm ischemia (WI) time, complications associated with ECD kidneys are unavoidable consequences. Ischemia and reperfusion injury (IRI) is also relevant to the discussion, a process causing great disturbance to the natural performance and physiological processes of the kidney allograft. The effects of total ischemia time are shown to be exacerbated by other donor characteristics such as age. This multifactorial complexity requires innovation in therapeutic approaches to improve ischemia time in organ transplantation.

Normothermic machine perfusion (NMP), often referred to as ex-vivo normothermic perfusion (EVNP), or normothermic ex-vivo kidney perfusion (NEVKP), has emerged in a growing conversation surrounding organ preservation and transplantation techniques with the goal of improving patient and clinical outcomes. It has been studied as a possible tool to mitigate ECD-associated complications. This perfusion method holds further possibilities with respect to the procurement, transplantation, and post-transplantation success. As a preservation technique, NMP mitigates the presentation of ischemia-related complications often seen in grafts preserved via static cold storage (SCS). Thus, NMP can promote kidney function and suppress consequences related to SCS. As a potential assessment tool, NMP may serve as a more accurate screening mechanism to assess graft potential and viability, and lower the number of discarded kidneys at risk for delayed graft function (DGF) and graft failure. Beyond these boundaries, NMP may also serve as a reconditioning mechanism. Given these possibilities, a better understanding of not only NMP, but its relation to involved pre- and post-transplantation complications will allow for improved patient outcome within multiple domains.

### BRIEF SUMMARY OF THE INVENTION

One aspect of the disclosure is a human kidney perfusion system comprising: a first container configured to hold a first human kidney; a second container configured to hold a second human kidney; a first centrifugal pump configured to deliver perfusate comprising red blood cells to the first container; a second centrifugal pump configured to deliver perfusate comprising red blood cells to the second container; a first oxygenator in fluid communication with the first container configured to oxygenate the perfusate pumped to the first container; and a second oxygenator in fluid communication with the second container configured to oxygenate the perfusate pumped to the second container.

Another aspect of the disclosure is a method of testing a pharmaceutical composition in a human kidney perfusion model, the method comprising: feeding perfusate comprising red blood cells and the pharmaceutical composition to a first human kidney held within a first container; feeding perfusate comprising red blood cells to a second human kidney held within a second container; analyzing fluid and/or tissue obtained from the first human kidney after contact with the perfusate; and analyzing fluid and/or tissue obtained from the second human kidney after contact with the perfusate. This method can be accomplished using the perfusion system disclosed elsewhere herein.

In one embodiment, a human kidney normothermic perfusion system is configured to perfuse kidney allografts from a single donor.

In one embodiment, Perfusion Regulated Organ Therapeutics with Enhanced Controlled Testing (PROTECT) is used to pump arterial blood to a pair of kidneys.

### BRIEF DESCRIPTION OF THE DRAWINGS

FIG. 1 is an illustration of a human kidney perfusion system of the present disclosure;

FIG. 2 is an illustration of a human kidney undergoing normothermic perfusion; and

FIG. 3 is another illustration of the human kidney undergoing normothermic perfusion with production of urine.

### DETAILED DESCRIPTION

The increased utilization of renal grafts for transplantation requires optimization of pre-transplant organ assessment strategies. Current methods to accept an organ for transplantation lack overall predictive power. Despite the ongoing research of NMP, further studies are necessary to establish standardizable protocol before common practice. Consensus has not been reached on the most advantageous parameters for implementing NMP into widespread clinical practice. Consensus has not been reached on the most advantageous parameters for implementing NMP into widespread clinical practice. A conclusive understanding of the key biomarkers to analyze during NMP as well as the composition of perfusate solutions and durations is also lacking. The scarcity of NMP kidney studies in the United States highlights the further need for research in this domain. Further studies are also needed to enhance our understanding of NMP in the context of interrelated complications such as kidney IRI. Establishing consensus and gaining deeper insight into both issues will progress the future of nephrology and therapeutic practice.

Currently there is no nationally-available NMP kidney system that can replicate the improved kidney transplant outcomes. Thus, such a platform for kidneys is of great national importance to assess true organ viability, and it could allow for rehabilitation and rescue of those organs before transplant. Furthermore, there is no available NMP kidney system that allows for two kidneys to undergo NMP at once on separate perfusion circuits. As described herein, an NMP kidney system that maintains two kidneys at once on separate perfusion circuits would allow for tests on one kidney while the other can be used as an internal control.

### Perfusion System

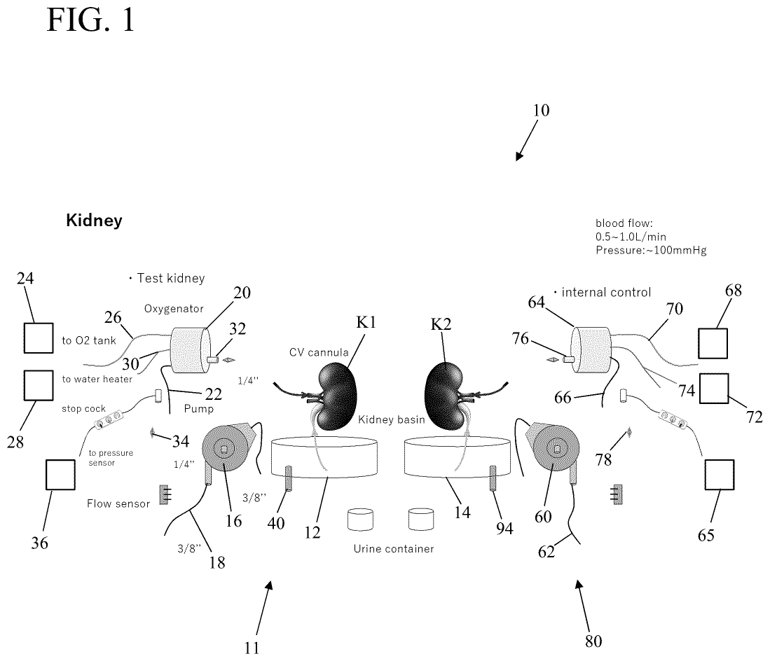

Referring to FIG. 1, a system for human kidney perfusion is generally indicated at 10. In general, the system is configured to perform normothermic kidney perfusion for use in clinical testing and preserving kidney transplantations. As such, in one aspect, the system 10 incorporates an improved testing platform configured with an internal control through simultaneous perfusion of a pair of kidneys. Therefore, the system 10 comprises a human kidney perfusion model with dual perfusate supply channels, permitting two kidneys from the same donor (or different donors) to be perfused simultaneously but separately for rigorously controlled therapeutic testing. As a result, the system 10 is configured to prevent cold ischemia injury to a deceased donor kidney through perfusion treatment. In one embodiment, the system 10 comprises a Perfusion Regulated Organ Therapeutics with Enhanced Controlled Testing (PROTECT).

Referring still to FIG. 1, the system 10 generally comprises a first container 12 configured to receive and support a first human kidney K1, and a second container 14 configured to receive and support a second a human kidney K2. In one embodiment, the first and second kidneys K1, K2 comprise a pair of kidneys from the same human donor. It will be understood, however, that in one or more other embodiments the containers 12, 14 could receive kidneys from two different donors without departing from the scope of the disclosure. A first fluid mover (e.g., pump 16) is configured to move (e.g., pump) fluid (perfusate—e.g., red blood cells/blood, test fluid, nutrients, medication, etc.) to the first container 12. Although the pump 16 is shown as the fluid mover, other types of suitable fluid movers for feeding perfusate to the first container 12 may be used. In the illustrated embodiment, first tubing 18 is fluidly connected to the first pump 16 and extends in both an upstream direction and a downstream direction. The first pump 16 is configured to pump perfusate through the tubing in the downstream direction from the pump. In the illustrated embodiment, the first pump 16 comprises a centrifugal pump operable to move perfusate through the first pump tubing 18 in the downstream direction. For example, the pump 16 may be a centrifugal pump from an extracorporcal membrane oxygenation (ECMO) machine with a modified circuit. Such a centrifugal pump is suitable for pumping the perfusate described herein. In one or more other embodiments, the pump 16 may be a different type of pump suitable for functioning as described herein.

A first oxygenator 20 is in fluid communication with a downstream section of the first pump tubing 18 via a first deoxygenated blood line 22. A first oxygen tank 24 is connected to the first oxygenator 20 via a first oxygen line 26, and a first heater 28 is connected to the first oxygenator by a heat conduit 30. In one embodiment, the first heater 28 comprises a water heater configured to deliver heated water through the heat conduit 30 to transfer heat from the heated water to the first oxygenator 20 for warming the perfusate flowing through the oxygenator. A first oxygenated perfusate line 32 extends from the oxygenator 20 to the first container 12. The first oxygenator 20 is configured to warm and oxygenate the deoxygenated red blood cells in the perfusate being pumped through the first pump tubing 18 by the first pump 16, as will be explained in greater detail below. In the illustrated embodiment, a connector 34 connects the first deoxygenated perfusate line 22 to the first pump tubing 18. However, in one or more other embodiments the first deoxygenated perfusate line 22 could comprise a segment of the first pump tubing 18 or a segment integrally formed with the first pump tubing without departing from a scope of the disclosure. In the illustrated embodiment, the first oxygenator 20 is in fluid communication with the first container 12 but is not in fluid communication with the second container 14. In one or more other embodiments, the first oxygenator 20 may be in fluid communication with both containers 12, 14 for delivering oxygenated perfusate to both containers.

In the illustrated embodiment, a pressure sensor 36 is connected to the system 10 to monitor pressure in a first perfusion circuit 11. In one embodiment, the first pump tubing 18, first deoxygenated perfusate line 22, oxygenator 20, first oxygenated perfusate line 32, and first container 12 comprises the first perfusion circuit 11. It will be understood that the first perfusion circuit 11 may comprise more or less components without departing from the scope of the disclosure.

The first pump 16 is configured to circulate perfusate (e.g., red blood cells/blood, test fluid, nutrients, medication, etc.) through the first perfusion circuit 11 to perform perfusion treatment on the first kidney K1 in the first container 12. Operation of the first pump 16 delivers fluid received from an upstream section 40 of the first pump tubing 18 through the downstream section of the first pump tubing. The fluid, and in particular, the perfusate in the upstream section of the first pump tubing 18 contains deoxygenated red blood cells as it is expelled from the outlet of the first container 12. The first pump 16 forces the perfusate in the first pump tubing 18 in the downstream direction toward the oxygenator 20. As such, fluid that is directed from the first pump tubing 18 through the first deoxygenated perfusate line 22 travels to the oxygenator 20 where the deoxygenated red blood cells in the perfusate are warmed and oxygenated. The oxygenated red blood cells then delivered through the oxygenated perfusate line 32 to the first kidney K1 in the first container 12. For example, the oxygenated perfusate line may be connected to single or double renal arteries of the first kidney K1. Perfusate in the first container 12 can then be drained through the outlet in the container and directed back to the first pump 16 for recirculation through the circuit 11. As will be understood, the perfusate will be drained from a renal vein of the first kidney K1. Thus, the pump 16 will pump perfusate to the oxygenator 20 and then the renal artery of the first kidney K1. Venous perfusate will then pass from the renal vein of the first kidney K1 directly into the first container 12 to complete the circuit. Therefore, the first perfusion circuit 11 is configured to simulate in-vivo conditions with allogeneic red blood cell transfusion through the pump 16.

This circulation of perfusate comprising red blood cells to and from the first kidney K1 in the first container 12 may function to keep the first kidney metabolically active in situations where the kidney is being treated prior to transplantation. Additionally, the supply of perfusate comprising red blood cells to the first kidney K1 in the first container 12 can be used to facilitate analysis of perfusate, blood, urine, and/or tissue obtained from the first kidney K1 after contact with the perfusate comprising red blood cells pumped to the first kidney. Still other processes can be performed with the first perfusion circuit 11.

Referring still to FIG. 1, a second fluid mover (e.g., pump 60) is configured to move (e.g., pump) perfusate comprising red blood cells to second container 14. In the illustrated embodiment, second pump tubing 62 is in fluid communication with the second pump 60 and extends in both an upstream and a downstream direction. The second pump 60 is configured to pump perfusate through the tubing 62 in the downstream direction from the pump. In the illustrated embodiment, the second pump 60 comprises a centrifugal pump for driving perfusate in the second pump tubing 62 in the downstream direction. For example, the pump 60 may be a centrifugal pump from an ECMO with a modified circuit. As with the first pump 16, the second pump 60 may be another type of suitable pump.

A second oxygenator 64 is in fluid communication with a downstream section of the second pump tubing 62 via a second deoxygenated perfusate line 66. A second oxygen tank 68 is connected to the second oxygenator 64 via a second oxygen line 70, and a second heater 72 is connected to the second oxygenator by a heat conduit 74. In one embodiment, the second heater 72 comprises a water heater configured for delivering heated water through the heat conduit 74 to transfer heat from the heated water to the second oxygenator 64 for warming the perfusate flowing through the oxygenator. A second oxygenated perfusate line 76 extends from the oxygenator 64 to the second container 14. The second oxygenator 64 is configured to warm and oxygenate the deoxygenated red blood cells in the perfusate being pumped through the second pump tubing 62 by the second pump 60, as will be explained in greater detail below. In the illustrated embodiment, a connector 78 connects the second deoxygenated perfusate line 66 to the second pump tubing 62. However, the second deoxygenated perfusate line 66 could comprise a segment of the second pump tubing 62 or a segment integrally formed with the second pump tubing without departing from a scope of the disclosure. In the illustrated embodiment, the second oxygenator 64 is in fluid communication with the second container 14 but is not in fluid communication with the first container 12. In one or more other embodiments, the second oxygenator 64 may be in fluid communication with both containers 12, 14 for delivering oxygenated blood to both containers.

A pressure sensor 65 may be connected to the system 10 to monitor pressure in a second perfusion circuit 80. In one embodiment, the second pump tubing 62, second deoxygenated perfusate line 66, oxygenator 64, second oxygenated perfusate line 76, and second container 14 comprises the second perfusion circuit 80. It will be understood that the second perfusion circuit 80 may comprise more or less components without departing from the scope of the disclosure.

The second pump 60 is configured to circulate perfusate (e.g., red blood cells/blood, control fluid, nutrients, medication, etc.) through the second perfusion circuit 80 to perform perfusion treatment on the second kidney K2 in the second container 14. Operation of the second pump 60 delivers perfusate received from an upstream section 94 of the second pump tubing 62 through the downstream section of the second pump tubing. The fluid, and in particular, the perfusate in the upstream section of the second pump tubing 62 contains deoxygenated red blood cells as it is expelled from the outlet of the second container 14. The second pump 60 forces the perfusate in the second pump tubing 62 in the downstream direction toward the oxygenator 64. As such, perfusate that is directed from the second pump tubing 62 through the second deoxygenated perfusate line 66 travels to the oxygenator 64 where the deoxygenated perfusate comprising red blood cells in the fluid is warmed and oxygenated. The oxygenated red blood cells are then delivered through the oxygenated perfusate line 76 to the second kidney K2 in the second container 14. For example, the oxygenated perfusate line may be connected to a renal artery of the second kidney K2. Perfusate in the second container 14 can then be drained through the outlet in the container and directed back to the second pump 60 for recirculation through the circuit 80. As will be understood, the perfusate will be drained from a renal vein of the second kidney K2. Thus, the pump 60 will pump perfusate to the oxygenator 64 and then the renal artery of the second kidney K2. Venous perfusate will then pass from the renal vein of the second kidney K2 directly into the second container 14 to complete the circuit. Therefore, the second perfusion circuit 80 is configured to simulate in-vivo conditions with allogeneic red blood cell transfusion through the pump 60.

This circulation of perfusate comprising red blood cells to and from the second kidney K2 in the second container 14 may function to maintain the second kidney in a control state in situations where the kidney is being treated prior to transplantation. Additionally, the supply of perfusate comprising red blood cells to the second kidney K2 in the second container 14 can be used to facilitate analysis of perfusate, blood, urine and/or tissue obtained from the second kidney after contact with the perfusate comprising red blood cells pumped to the second kidney. Still other processes can be performed with the second perfusion circuit 80.

### Method of Testing a Pharmaceutical Composition

The disclosure is further directed to a method of testing a pharmaceutical composition in a human kidney perfusion model, the method comprising: feeding perfusate including red blood cells and the pharmaceutical composition to a first human kidney held within a first container; feeding perfusate including red blood cells to a second human kidney held within a second container; analyzing fluid and/or tissue obtained from the first human kidney after contact with the perfusate; and analyzing fluid and/or tissue obtained from the second human kidney after contact with the perfusate. This method can be accomplished using the disclosed perfusion system.

The first and second human kidneys can be fed with the perfusate simultaneously for rigorously controlled therapeutic testing to prevent cold ischemia injury. The method can further comprise pumping the perfusate comprising red blood cells and the pharmaceutical composition through a first perfusion circuit including the first container, a first pump tubing, a first oxygenator, and a first oxygenated perfusate line. The method can further comprise pumping the perfusate comprising red blood cells through a second perfusion circuit separate from the first perfusion circuit, the second perfusion circuit including the second container, a second pump tubing, a second oxygenator, and a second oxygenated perfusate line. The method can further comprise monitoring pressure in the first and second perfusion circuits. The method can further comprise oxygenating the perfusate fed to the first and second containers. The method can further comprise warming the perfusate fed to the first and second containers. The perfusate can be warmed to from about 35° C. to about 37° C.

The first and second human kidneys can be from a single human donor or from two different human donors. The first and second human kidneys can be disqualified from transplant into a human patient, such as due to assessed kidney quality and/or predicted transplant outcome.

The perfusate can flow through the first and second human kidneys at a rate of about 0.5 to about 1.0 L per minute. The perfusate can flow through the first and second human kidneys with a pressure from about 65 mm Hg to about 75 mm Hg.

The perfusate can further comprise one or more additional components known in the art to be used in perfusate. Exemplary components include, but are not limited to water, Normal Saline solution, Ringer's solution, Ringers lactate, STEEN solution, Williams' Medium E, TPN nutriflex, hemoglobin-based oxygen carrier, alpha-1 antitrypsin, heparin, insulin, cefazolin, augmentin, creatinine, albumin, amino acids, urine recirculation, dextran, dexamethasone, mannitol, glucose, calcium gluconate, magnesium sulfate, sodium bicarbonate, trisodium phosphate, sodium chloride, potassium chloride, and multivitamins and other nutrients.

The pharmaceutical composition can comprise at least one of a small molecule, a nucleic acid, an enzyme, an antibody, a contrast agent, a gene therapy, and a cell therapy. The small molecule can be a compound or drug. The nucleic acid can be any single or double stranded DNA or RNA construct such as, for example, an RNAi construct. The enzyme can be an enzyme to contribute to a metabolic process of the kidney or improve the chance of kidney transplant success. An exemplary enzyme is an alpha-galactosidase enzyme used to remove blood group antigens. The antibody can be a monoclonal or polyclonal antibody. The contrast agent can be used for MRI imaging and can be radiolabeled. The gene therapy can be designed to modify the genome in one or more cells of the subject and can involve, for example, a bioengineered virus and/or CRISPR-Cas9 technology known in the art. Exemplary cell therapies include mesenchymal stromal cells (MSCs) and induced pluripotent stem cells (iPSCs).

The pharmaceutical composition can further comprise a carrier or vehicle used to solubilize the component described above. Exemplary vehicles include, but are not limited to, water, saline, DMSO, ethanol, and other solvents.

The perfusate comprising red blood cells fed to the second human kidney can further comprise the vehicle used in the pharmaceutical composition. The perfusates fed to the first and second human kidneys can be identical except for one component in the pharmaceutical composition as described above (i.e. the perfusate fed to the second human kidney is a control for the perfusate fed to the first human kidney).

The pharmaceutical composition can be tested to identify a composition that can improve kidney function, transplantation success, and/or increase the time of successful perfusion. The method can be used to identify a pharmaceutical composition that can improve kidney quality assessment, kidney preservation, and/or kidney repair or reconditioning.

The method can further comprise comparing the results of analyzing the fluid and/or tissues obtained from the first and second human kidneys to determine the effectiveness of the pharmaceutical composition.

The analyzed fluid can be perfusate, urine, and/or blood.

The analyzing can comprise at least one selected from the group consisting of measuring the level of at least one analyte in the fluid and/or tissue, imaging the fluid and/or tissue, histopathological assessment of the tissue, and measuring the volume and/or weight of the fluid and/or tissue.

The analyte can be selected from the list consisting of creatinine, aspartate aminotransferase (AST), lactate dehydrogenase (LDH), kidney injury molecule-1 (KIM-1), neutrophil gelatinase-associated lipocalin (NGAL), N-acetyl-β-D glucosaminidase (NAG), thiobarbituric acid reactive substance, serum creatinine, pCO2, pO2, bicarbonate, electrolytes (Na+, K+, Cl—, and ionized Ca2+), and combinations thereof.

The analyzing can further comprise determining calculated CrCl (creatinine clearance). The histopathological assessment of the tissue can comprise at least one selected from the group consisting of assessing the degree of cortical necrosis, tubular injury, and renal tubuloepithelial cell death. The imaging can comprise MRI imaging.

The analyzing can further comprise measuring at least one selected from the group consisting of pH, RBF (renal blood flow), intrarenal resistance, total urine output, BGA (blood gas analysis), hemodynamic stability, kidney function, histologic integrity, arterial flow, histological assessment, serum sample analysis, hourly blood and/or urine sample analysis, kidney weight, and urine output.

The analyzing can further comprise at least one outcome measure selected from the list consisting of RBF, mean arterial pressure, tubular function, graft injury/survival/function, patient survival/status, EVNP assessment score, rates of DGF (need for dialysis in first week post-transplant), incidence of PNF, duration of DGF, renal function, urine recirculation levels, renal perfusion/flow, oxygen consumption, and diuresis.

The method can be completed within about 1 hour, about 2 hours, about 3 hours, about 4 hours, about 6 hours, about 12 hours, about 18 hours, about 24 hours, about 48 hours, about a week, or about a month.

### NMP Techniques to Optimize

The methods disclosed herein can be used to determine optimized conditions to use in NMP. Exemplary conditions to test include temperature, perfusate composition and parameters, oxygenation conditions, when to begin NMP and its duration, biomarkers to analyze, and types of imaging to use in analysis.

### Temperature Conditions

NMP is of significant value to the future of kidney transplantation because it provides near-physiologic conditions (35° C.-37° C.). Differing from SCS and HMP (4° C.), NMP can support optimal metabolic function by ensuring the replenishment of ATP and preventing ischemic injury. In sub-physiological temperature conditions, the depletion of ATP by means of reduced metabolic function which in turn, slows or inactivates additional cellular processes.

### Perfusate Composition

While perfusate composition varies amongst currently published protocols, major commonalities still exist. Typically, the perfusate for NMP contains an oxygen carrier, priming solution, fluid replacement, a colloid, nutrient supplementation, anticoagulant, and protective additives. In a porcine kidney model, use of varying volumes of red blood cells (RBCs) ranging from 350 to 170 ml among four different perfusion solution groups undergoing 7 h of NMP has been shown. Variation in the levels of injury marker N-acetyl-β-D glucosaminidase (NAG), NMP flow patterns, and thiobarbituric acid reactive substance levels amongst each perfusion group has been assessed. It has been shown previously that varying perfusate composition can affect interpretation of perfusion parameters.

NMP can also be conducted with or without urine recirculation. In studies demonstrating feasibility of urine recirculation, Ringer's lactate solution has been the chosen solution for fluid replacement. It has been shown that urine recirculation provides an effective method of maintaining perfusate homeostasis. On the other hand, NMP has also been utilized on porcine grafts for 16 h without the use of urine recirculation.

Kidneys undergoing NMP are primarily perfused with a plasma-free RBC solution. Different volumes of RBC solution may be used in different models. An optional alternative to an RBC solution is an artificial oxygen carrier, although not as commonly used. The use of hemoglobin-based oxygen carrier (HBOC-201), a bovine-derived alternative to the use of RBC units, has been demonstrated as a suitable artificial alternative based on a porcine kidney model undergoing ex-vivo SNMP (subnormothermic machine perfusion).

Perfusate composition may also involve the delivery of a therapeutic agent during perfusion. In a porcine kidney perfusion model, the feasibility of introducing alpha-1 antitrypsin (AAT) during NMP after the priming solution begins circulating through the perfusion circuit has been demonstrated. A protease inhibitor, AAT, may offer a potential therapeutic mechanism to respond to kidney inflammation and immune activation from IRI.

Perfusate composition may also depend on the purpose of NMP. For purposes of assessment, preservation, or repair, the perfusate contents are often chosen accordingly. The variability of perfusate contents may also be chosen differently based on whether NMP is preceded by other storage mechanisms, such as SCS or HMP, prior to transplantation.

Possible perfusate compositions include:
1) Ringer's solution, RBC (red blood cell) 1 unit, mannitol 10%, dexamethasone 8 mg, heparin, sodium bicarbonate 8.4%, nutrient solution, insulin, and multivitamins infused at 20 ml/h
2) RBC 202 ml, Ringer's solution, Albumin 22.5 g, insulin and amino acids, heparin, dexamethasone 10 mg, calcium gluconate 10%, and sodium bicarbonate 8.4%
3) RBC 1 unit, 5% human albumin 250 ml, Ringer's solution/urine recirculation, TPN nutriflex, mannitol 10%, calcium gluconate 10%, and sodium bicarbonate 8.4% 5-15 ml
4) RBC 125 ml, STEEN solution, Ringer's solution, amino acids, heparin, dexamethasone 10 mg, calcium gluconate 10%, and sodium bicarbonate 8.4%
5) 350 ml autologous RBCs and 500 ml Williams' Medium E, 10 ml augmentin, and 1000 μmol/L creatinine
6) 350 ml autologous RBCs, 250 ml NaCl 0.9%, 2 ml augmentin, 1000 μmol/L creatinine, 21 ml NaHCO₃8.4%, 1.4 ml MgSO₄, 6.5 ml calcium gluconate, 11 ml glucose, 2.5 ml KCl 74.6 mg/ml, 0.2 ml Na₃PO₄, 90 ml sterile water, and 200 ml albumin
7) 170 ml autologous RBCs, 290 ml Ringers lactate, 2 ml augmentin, 1000 μmol/l creatinine, 27 ml NaHCO₃, 10 ml mannitol, 3 ml heparin, and 3.75 mg dexamethasone
8) 290 ml autologous RBCs, 300 ml NaCl, 8 ml augmentin, 1000 μmol/l creatinine, 8 ml NaHCO₃, 10 mg mannitol, 4.8 ml calcium gluconate, 9.6 ml glucose, and 8 IU insulin
9) RBC 544 ml, sodium bicarbonate 8.4%, calcium gluconate 10%, mannitol 32 mg, sodium chloride 0.9%, creatinine 160 mg, and glucose 5%
10) dextran/albumin 215 ml, RBC 400 ml, insulin and amino acids, calcium gluconate 10%, heparin, cefazolin 400 mg, and Ringer's solution

### Oxygenation Conditions

Suitable oxygenation conditions are a fundamental requisite in maintaining cellular metabolic function and preventing injurious renal complication. Although ideal conditions and the effect of oxygenation conditions on kidney graft function remain unclear, many NMP systems utilize a gas mixture of 95% O₂-5% CO₂ or even 100% O₂. These high levels of oxygen delivered throughout NMP are a means to maintain blood pH through the duration of perfusion. Although discussion regarding oxygen partial pressures varies as some studies suggest maintaining oxygen partial pressures (P_{O2}) within physiological limits is preferential, many current studies report successful use of physiological oxygen concentrations. There is yet to be consensus on whether physiological oxygenation levels are preferential to supra-physiological conditions. Blood gas analysis and arterial pressures (P_{O2} and P_{CO2}) are among the most reported parameters monitored in recent studies. Reducing oxygen level throughout perfusion to assess its effect on kidney function provides evidence that although oxygen kinetics was altered, reduced oxygen levels did not disrupt tubular function in a porcine ex-vivo perfusion model.

### When to Begin NMP and Perfusion Duration

The initiation and duration of NMP is another factor often tailored to the purposes of NMP (kidney assessment, preservation, and repair). Several recent studies demonstrated an NMP model in which a shorter perfusion duration is utilized immediately preceding transplantation at the recipient transplant site. This model of NMP allows for pre-transplant assessment in proximity to the potential recipient. In contrast, prolonged NMP as an alternative to traditional SCS has also been studied as a viable tool on its own. Studying perfusion in porcine DCD kidney grafts, it has been shown that grafts undergoing NMP alone showed significantly lower peak serum creatinine levels relative to the SCS only group at post-operative day 3 after transplantation. Furthermore, a comparison between the groups undergoing 16 h NMP, 8 h NMP, and 15 h SCS with 1 h NMP revealed that grafts with 16 h of NMP exhibited a trend of lower serum creatinine levels following transplantation when compared to other groups (SCS 16 h; 8 h SCS+8 h NMP, and 15 hSCS+1 h NMP). These observations suggest not only that NMP has benefits as a sole alternative to SCS, but also that a prolonged duration of NMP may be advantageous.

Exemplary durations of NMP include: 1 hour, 1-3 hours, 3 hours, 7 hours, 8 hours, 16 hours, and 24 hours with urine recirculation and 7.7+/1.5 hours with Ringer's solution 7) 1-3 hours

### Perfusion Parameters and Biomarkers

Biochemical injury markers are an important source of information for the assessment of kidney function and performance both during and after NMP. Measurements of relevant biomarkers can be taken from perfusate, blood, or urine samples depending on the marker of interest. These measurements provide informative values of kidney quality and serve as an aid in the decision-making process for acceptability of transplant.

Various markers of injury have been reported as assessment tools in kidney perfusion, and thus there is not a unified consensus on which are most important to observe in relation to kidney NMP. Although consensus is lacking, several notable biomarkers are frequently reported in literature. Aspartate aminotransferase (AST), lactate dehydrogenase (LDH), kidney injury molecule-1 (KIM-1), and neutrophil gelatinase-associated lipocalin (NGAL) have important applications to kidney NMP and assessment of graft function. KIM-1 and NGAL are two of the most recognized markers of kidney injury. NGAL is an injury marker for acute renal injury. Similarly, KIM-1 has been shown as a marker for human renal proximal tubule injury. A study demonstrated that based on urine collection from 32 adult patients with various renal complications, KIM-1 had increased expression levels in those with ischemic acute tubule necrosis (ATN) than those with other forms of AKI. Both KIM-1 and NGAL are markers measured via a urine sample. Another study used a human kidney NMP model to show that NGAL and KIM-1 had a clear decrease as the duration of NMP increased. The extensive use of KIM-1 and NGAL as an assessment measure in NMP protocols allows for a reliable understanding of kidney perfusion. AST and LDH are also widely common markers of cellular damage and a means of assessing tissue injury. In a porcine perfusion model, it has been shown that (1) levels of AST and LDH were below detectable levels at each hourly perfusion assessment, and (2) although increased in all groups, AST and LDH remained lowest during NMP in all groups. Perfusion solution creatinine levels are also useful parameters for assessment of kidney function. During EVNMP, creatinine should be added to the circuit to enable subsequent quantification of creatinine clearance.

Various parameters that have been monitored include, but are not limited to, RBF (renal blood flow), intrarenal resistance, total urine output, BGA (blood gas analysis), hemodynamic stability, kidney function, histologic integrity, arterial flow, histological assessment (optionally on post-operative day 8), serum sample analysis, hourly blood and/or urine sample analysis, LDH (lactate dehydrogenase), ASAT (aspartate aminotransferase), NAG (N-acetyl-β-D glucosaminidase), MDA (malondialdehyde), serum creatinine, creatinine clearance, change in renal weight, urine output, perfusate sample culture, and other biomarkers.

Various outcome measures that have been used include RBF, mean arterial pressure, tubular function, graft survival (incidence of PNF/DGF-primary nonfunction/delayed graft function), patient survival/status at 12 months, EVNP (ex-vivo normothermic perfusion) assessment score relying on renal blood flow and urine output thresholds, renal function during EVNP, rates of DGF (need for dialysis in first week post-transplant), incidence of PNF, duration of DGF, functional DGF, renal function, assessment of graft injury markers, survival (measured 8 days following transplantation), assessment of renal function and graft injury, graft survival, urine recirculation levels, quantification of perfusate biomarker, graft function (serum creatinine levels after autotransplantation), levels of injury markers (AST+LDH) during ex-vivo perfusion, renal perfusion/flow during 3 hours of NMP (assessed every 1 hour) creatinine clearance levels, oxygen consumption, and diuresis.

NMP in Combination with Diagnostic and Functional Imaging

As the quantification of biomarkers and monitoring of parameters throughout NMP is pivotal to assess, imaging techniques may also enhance understanding of kidney physiology during perfusion. Various diagnostic and functional imaging techniques can be used in association with NMP. The first study to utilize magnetic resonance imaging (MRI) techniques to understand renal perfusate flow distribution utilized arterial spin labeling (ASL) using water molecules as the contrast agent to visualize flow distribution at intervals of 15 min for the entire duration of the 3 h NMP protocol, assessing both porcine and human kidneys. This study showed that beginning perfusion, kidneys were centrally perfused while the cortex reached a physiological perfusion level after hours 1 and 2. In this circumstance, functional imaging in conjunction with NMP must be utilized with caution as results might not always reflect physiological accuracy. Nonetheless, MRI imaging allows for time-interval comparisons of physiological changes throughout the duration of NMP.

Diagnostic and functional imaging in association with NMP is a clinical avenue that must be explored in more detail before its findings provide substantial physiological evidence. These techniques offer major potential as visualization tools for understanding the mechanism of NMP on physiological kidney processes, especially as they are non-invasive. Applying these to a standard NMP protocol may help optimize the assessment process and decision-making model for determining viable kidney donations acceptable for transplantation.

An NMP setup with MRI compatibility also offers a unique clinical mechanism for assessing intrarenal physiology throughout the progression of NMP. Increasing the combined use of NMP and diagnostic imaging techniques holds great potential in achieving consensus on relevant biomarkers, optimal protocol, and the most important parameters to monitor. Further investigations are needed to explore the potential of NMP with related therapeutic modalities, including antioxidant therapy and nutrients, administration of MSCs, and therapeutic gases during kidney perfusion. These avenues should be prioritized as future focus points for better understanding of NMP and development of therapeutic interventions.

### Areas of Study and Clinical Implications

### Pathophysiology of Ischemia and Reperfusion Injury

To optimize therapeutic approaches in mitigating kidney IRI and its resulting transplant consequences, it is important to acknowledge its complexity. Ischemia is the discontinuance of blood flow to a particular organ or tissue and reperfusion injury is the organ damage succeeding re-establishment of perfusion. While ischemia and reperfusion injury are not solely associated with organ transplantation and post-operative kidney outcomes, substantial research exists to strengthen knowledge of this connection. This research focuses on a goal to understand the molecular and cellular effects of IRI. Most notably, IRI affects total protein (TP) levels thereby resulting in a lack of ATP re-synthesis which cascades into energy depletion in the injured tissue or organ. This ATP depletion is preceded by an abnormally high production of reactive oxygen species (ROS) within the injured tissue. An additional hallmark of IRI relates to the distinct isoforms of nitric oxide synthase (NOS), the enzyme responsible for the synthesis of nitric oxide (NO). Its isoform inducible NOS (iNOS) produces free radicals in hypoxic cells and works as a toxic agent that elicits an adverse kidney response while endothelial NOS (cNOS) causes vasodilation and serves as a reno-protective enzyme. Previously, it has been demonstrated that hypothermic machine perfusion (HMP) is better than SCS at improving microcirculation via stress-induced NO release by increasing activation of eNOS phosphorylation, in turn showing that machine perfusion is a valuable tool for preserving the reno-protective capability of the NO signaling pathway. The cellular effects of IRI are also marked by a substantial and adverse increase in intracellular ion levels of calcium (Ca2+) and sodium (Na+). Additional research is concerned with the influence of regulated cell death pathways on tubular cell death in IRI, such as ferroptosis. Remaining largely unknown, these regulated cell death pathways are focus points for ongoing research surrounding kidney IRI and transplant outcomes.

### Consequences of Ischemia and Reperfusion Injury on Kidney Transplant Outcomes

IRI is a significant factor in kidney transplant outcomes. IRI-specific implications on the kidney, of the most serious, include acute kidney injury (AKI), delayed graft function (DGF), and damaging activation of innate immunity. DGF is caused by the combination of ischemia and reperfusion. Ischemia makes renal cells vulnerable to oxidative stress. Reperfusion supplies oxygen to the kidney, but it causes inflammation via a white blood cell and cytokine response, and induces oxidative stress in renal cells. It has been demonstrated that DGF is preceded by a post-reperfusion metabolic collapse using a comparative analysis of biopsied human tissue and arterio-venous blood sample before and after reperfusion. Episodes of AKI following IRI can very rapidly progress to chronic kidney disease (CKD), and this is a complex, yet important mechanistic transition that requires identification and exploration for potential therapeutic innovations with NMP. Activation of innate immunity, which plays a crucial role in inflammation, is guided by toll-like receptors (TLRs). TLRs are upregulated by AKI, but they also recognize endogenous molecules known as damage associated molecular patterns (DAMPs) that are released by cells of injured tissues. Improper activation of innate immunity is a complicated IRI-related consequence to control with pre-transplant intervention; however, growing investigation is emerging into potential therapeutic strategies.

Mitigation of Renal Consequences from Ischemia and Reperfusion Injury

There are multiple options to minimize allograft injury throughout the transplantation process with respect to donor and recipient management prior to transplantation and graft management during the storage/preservation period. This includes graft reconditioning which consists of a determined time of WI for the donor graft to undergo, adequate perfusion of the graft, and ensured proper hydration of the recipient to prevent hypovolemia post-transplant.

Specific benefits of NMP as they relate to IRI and other techniques such as SCS or HMP include decreasing CIT, restoring ATP depletion, and decreasing apoptotic and oxidative distress markers. It has been demonstrated that porcine kidneys with 8 h NMP had lower serum creatinine levels and lower tubular injury and inflammation scores following perfusion compared to the groups without preservation or static cold storage. Results also suggest the possibility of additional strategies administered in concordance with NMP. For example, it has been demonstrated that the use of mesenchymal stromal cells (MSCs) on a porcine kidney model undergoing 240 minutes of NMP, concluding that administration of these cells during perfusion had no harmful effect on other perfusion factors. The use of MSCs with NMP is noteworthy in that these cells induce anti-inflammatory effects directly at the site of tissue injury, potentially helping to mitigate the detrimental effects of kidney IRI. In a similar therapeutic strategy, the use of Multipotent Adult Progenitor Cells (MAPC) has been demonstrated as a potential tool to modulate the effects of IRI in declined human kidneys that were not transplanted. Outcome measures occurred ex-vivo and compared to the control group, kidneys receiving MAPC treatment demonstrated improved urine output, decreased levels of the injury marker, NGAL, and downregulation of interleukin (IL)-1β.42 These types of cellular therapy techniques are pivotal for advancing the clinical understanding of kidney NMP. The assessment of metabolic homeostasis and the emphasis on optimal metabolic support during NMP is of pivotal importance to ensuring graft quality.

### Potential Clinical Use of Kidney NMP

IRI is a significant barrier to graft success and longevity. NMP may offer potential mitigation of this issue in a variety of manners. Namely, NMP presents as a potential technique for (1) kidney quality assessment, (2) kidney preservation, and (3) kidney repair or reconditioning. The purpose of NMP guides its use and often informs the duration of perfusion.

### Kidney Quality Assessment

Assessment of organ quality is an essential step in deciding the acceptability and viability of the donor kidney. This ensures best graft and patient outcomes. NMP reveals itself as a solution for assessment of quality in donor kidney grafts, especially as more ECD and DCD kidneys are being allocated. The feasibility of using NMP in this capacity has been demonstrated in association with a scoring system of kidney quality as a determinant of suitability before transplant. The score assessment developed takes into account renal blood flow thresholds, urine output assessment, and macroscopic appearance grading with a score of 1 designating highest graft quality and a score of 5 designating lowest graft quality. This application of a kidney quality scoring system in conjunction with NMP may help to lower the challenges that waiting recipients face in receiving a kidney transplant as well as a possible tool for transplant centers to utilize in assessing graft suitability. In turn, this may prevent unnecessary discard of kidneys.

In an additional study, a quality assessment score (QAS) was again used to determine which previously rejected human kidneys could be transplanted. Using this scoring system, kidneys identified with a score of 3 or less were deemed acceptable for transplant. The QAS was composed of mean renal blood flow, macroscopic assessment of perfusion, and urine output assessment. Human kidneys underwent 60 min of NMP, and five total kidneys were transplanted only one of which demonstrated DGF and had the highest QAS score (3) of those transplanted kidneys. As the only report that employed NMP on previously discarded human kidneys followed by transplantation, this study further demonstrates the capability of utilizing NMP as an assessment.

### Kidney Preservation

A potential alternative to SCS and HMP, NMP is further widely studied as a storage and preservation tool for grafts awaiting allocation and transplant. Studies have been done to compare the preservation potential of NMP to both HMP and SCS. Post-perfusion and transplant outcomes have been studied for porcine kidney models undergoing either 8 h of NMP or SCS. Comparison of kidney grafts amongst both groups showed that the NMP group exhibited acid-base parameters in stable homeostatic range as well as a high renal O₂ consumption. The injury markers, LDH and AST, were not seen in the NMP group, and there was no impact on patient survival amidst grafts that underwent perfusion prior to transplant. These findings suggest the potential of NMP as a replacement preservation option to SCS.

Using a porcine autotransplant model, it has also been shown that increasing duration of NMP while subsequently decreasing the duration of SCS resulted in lower peak serum creatinine levels for porcine kidneys in the 16 h NMP group compared to all other groups (16 h SCS, 15 h SCS+1 h NMP, and 8 h SCS+8 h NMP) following transplantation. This is a porcine autotransplant model in kidneys subjected to 30 min of warm ischemia at retrieval. It is debatable whether this is a good model of DCD kidney transplantation as the kidneys came from young, healthy pigs. Similarly, another study utilized three preservation groups where porcine kidneys were exposed to 30 min WI followed by 16 h of either SCS, HMP, or NMP. Results of the NMP group showed an improvement in creatinine clearance on post-operative day 3 while the SCS and HMP groups showed a significant decrease in creatinine clearance. Furthermore, the NMP group also demonstrated lower mean peak serum creatinine levels at an earlier post-transplant timepoint than that of either SCS or HMP groups. Amongst all three groups, tubular injury was equally low. These studies highlight the potential feasibility of NMP use for a duration longer than 8 h. Recently in the first randomized controlled 2 to compare traditional SCS (n=168) with SCS+1 h NMP (n=170) in DCD human kidney transplantations, it has been demonstrated that 1 h of perfusion does not reduce the occurrence of DGF.

### Kidney Repair and Reconditioning

Recent studies have also demonstrated the use of NMP subsequently following time periods of SCS and/or a period of WI as a means of showing its graft reconditioning capabilities. A single-center, UK-based study showed that levels of heat-shock protein-70 (HSP-70) and interleukin-6 (IL-6), two markers of cellular repair pathways, were upregulated in porcine kidneys undergoing 60 min of NMP thereby demonstrating repair capabilities. Another study demonstrated the use of NMP as a successor to WI time and SCS amongst three groups of porcine grafts undergoing 30 min induced WI time and 8 h SCS followed by either 1, 8, or 16 h of NMP. It has also been shown that of 22 discarded human kidneys undergoing 60 min NMP, 19 received an NMP assessment score of 1-4, and it was noted that these 19 kidneys would be potentially usable. These studies demonstrate the capability of using NMP for graft repair, but the duration of NMP for optimal repair ultimately remains unknown.

### Examples

The following non-limiting examples are provided to further illustrate the present invention.

### Kidney Procurement

Discarded human kidneys with negative serologies may be procured from either donation after brain death or donation after circulatory death donors. Allogeneic human blood may be further obtained from a blood bank. The discarded kidneys can be procured and remain in cold storage until the commencement of NMP, which will be performed at 37° C.

IRI with Normothermic Machine Perfusion Pump (Kidney PROTECT Pump)

Flow through the system 10 can be regulated by adjusting Hoffman clamps located along the pump tubing. Pressure monitoring can be performed on both the arterial limb of the circuit: mean arterial pressure can be titrated for a physiologic goal of 65-75 mmHg. Venous drainage from the kidney can then be collected by placing the kidney in a basin with an outflow port that is connected to the intake side of a centrifugal pump, thereby completing the circuit.

### Circuit Priming and Run

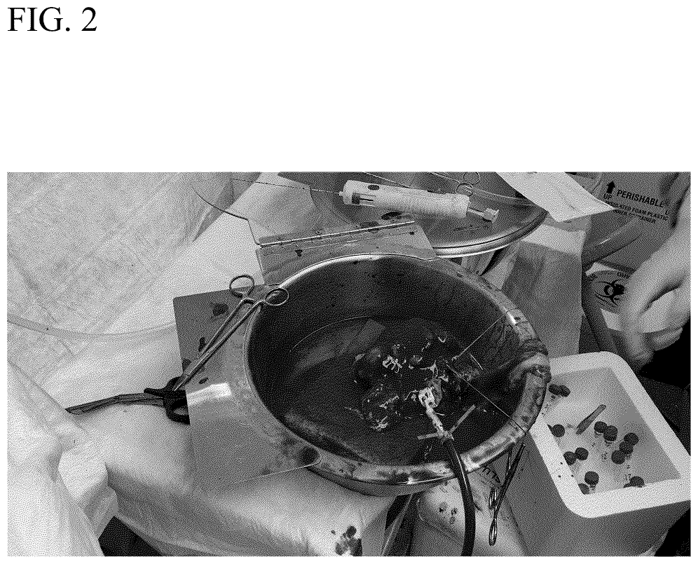

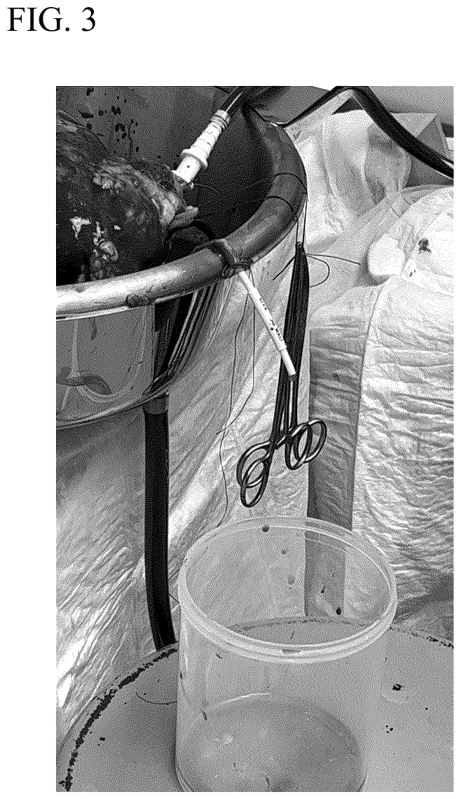

The renal artery of the kidney can be cannulated with a 6 Fr vascular cannula, if 2 renal arteries are encountered they can be connected to the vascular cannula using a Y connector, and the renal vein may be kept open. NMP can be performed with a combination of 3 U of donor's blood group matched packed RBC, to which Normal Saline solution (500 ml), 10% calcium gluconate (5 ml), 8.4% sodium bicarbonate (15 ml), sterile water (25 ml), and heparin (5000 U) is added. Urine can be collected from a ureter cannula (FIGS. 2 and 3).

### Outcome Measures for Effective NMP

Renal perfusion, biochemical, and histologic parameters can be collected at a pre-perfusion stage, and at 1 hour, 3 hours, and 6 hours post-perfusion with NMP. Perfusate pH, pCO2, pO2, bicarbonate, and electrolytes (Na+, K+, Cl—, and ionized Ca2+) levels can also be monitored. Adjustments to the sweep gas and blood flow are also completed, as necessary. The amount of urine output, perfusate, and urine concentrations of creatinine, KIM-1 and NGAL and calculated CrCl at 1 hour, 3 hours, and 6 hours post-perfusion are used to assess the effectiveness of the NMP. Additionally, a histopathological assessment including the degree of cortical necrosis, tubular injury, and renal tubuloepithelial cell death at 1 hour, 3 hour, and 6 hours post-perfusion will be used to assess the NMP.

When introducing elements of the present invention or the preferred embodiment(s) thereof, the articles “a”, “an”, “the” and “said” are intended to mean that there are one or more of the elements. The terms “comprising”, “including” and “having” are intended to be inclusive and mean that there may be additional elements other than the listed elements.

In view of the above, it will be seen that the several objects of the invention are achieved and other advantageous results attained.

As various changes could be made in the above apparatus, systems, and methods without departing from the scope of the invention, it is intended that all matter contained in the above description and shown in the accompanying drawings shall be interpreted as illustrative and not in a limiting sense.

What is claimed is:

1. A human kidney perfusion system comprising:
a first container configured to hold a first human kidney;
a second container configured to hold a second human kidney;
a first centrifugal pump configured to deliver a first perfusate comprising red blood cells to the first human kidney being held in the first container;
a second centrifugal pump configured to deliver a second perfusate comprising red blood cells to the second human kidney being held in the second container;
a first oxygenator in fluid communication with the first centrifugal pump and configured to oxygenate the first perfusate before the first perfusate is delivered to the first human kidney being held in the first container; and
a second oxygenator in fluid communication with the second centrifugal pump and configured to oxygenate the second perfusate before the second perfusate is delivered to the second human kidney being held in the second container.

2. The system of claim 1, wherein the first oxygenator is downstream of the first centrifugal pump, and wherein the second oxygenator is downstream of the second centrifugal pump.

3. The system of claim 1, further comprising:
first pump tubing fluidly connected to the first centrifugal pump;
a first deoxygenated perfusate line fluidly connected to a downstream end of the first pump tubing; and
a first oxygenated perfusate line extending downstream from the first oxygenator toward the first container,
wherein the first pump tubing, first oxygenator, first oxygenated perfusate line, and first container comprise a first perfusion circuit.

4. The system of claim 3, further comprising:
second pump tubing fluidly connected to the second centrifugal pump;
a second deoxygenated perfusate line fluidly connected to a downstream end of the second pump tubing; and
a second oxygenated perfusate line extending downstream from the second oxygenator toward the second container,
wherein the second pump tubing, second oxygenator, second oxygenated perfusate line, and second container comprise a second perfusion circuit separate from the first perfusion circuit permitting the first and second human kidneys to be pumped with perfusate simultaneously for controlled therapeutic testing.

5. The system of claim 4, wherein the first perfusion circuit comprises a first pressure sensor configured to monitor pressure in the first perfusion circuit, and wherein the second perfusion circuit comprises a second pressure sensor configured to monitor pressure in the second perfusion circuit.

6. The system of claim 1, further comprising a first heater operatively connected to the first oxygenator for warming the perfusate pumped to the first container, and a second heater operatively connected to the second oxygenator for warming the perfusate pumped to the second container.

7. A method of testing a pharmaceutical composition in a human kidney perfusion model, the method comprising:
feeding first perfusate comprising red blood cells and the pharmaceutical composition to a first human kidney held within a first container;
feeding second perfusate comprising red blood cells to a second human kidney held within a second container, wherein the second perfusate is free from the pharmaceutical composition;
analyzing at least one of fluid and tissue obtained from the first human kidney after said feeding first perfusate; and
analyzing at least one of fluid and tissue obtained from the second human kidney after said feeding second perfusate.

8. The method of claim 7, wherein said feeding first perfusate and said feeding second perfusate are performed simultaneously for controlled therapeutic testing to prevent cold ischemia injury.

9. The method of claim 8, wherein said feeding first perfusate comprises pumping the first perfusate through a first perfusion circuit including the first container, a first pump tubing, a first oxygenator, and a first oxygenated perfusate line.

10. The method of claim 9, wherein said feeding second perfusate comprises pumping the second perfusate through a second perfusion circuit separate from the first perfusion circuit, the second perfusion circuit including the second container, a second pump tubing, a second oxygenator, and a second oxygenated perfusate line.

11. The method of claim 10, further comprising monitoring pressure in the first and second perfusion circuits.

12. The method of claim 7, further comprising oxygenating the first and second perfusates fed to the respective first and second containers.

13. The method of claim 12, further comprising warming the perfusate fed to the first and second containers.

14. The method of claim 7, wherein the first and second human kidneys are from a single human donor.

15. The method of claim 7, wherein the perfusate flows through the first and second human kidneys at a rate of about 0.5 to about 1.0 L per minute.

16. The method of claim 7, wherein the fluid obtained from the first and/or second human kidney comprises at least one of perfusate, urine, and blood.

17. The method of claim 7, further comprising comparing results of the analysis of at least one of the fluid and tissues obtained from the respective first and second human kidneys to determine the effectiveness of the pharmaceutical composition.

18. The method of claim 7, wherein the pharmaceutical composition comprises at least one of a small molecule, a nucleic acid, an enzyme, an antibody, a contrast agent, a gene therapy, and a cell therapy.

19. The method of claim 7, wherein each of said analyzing at least one of fluid and tissue obtained from the first human kidney and the second human kidney comprises at least one of measuring the level of at least one analyte in the at least one of fluid and tissue, imaging the at least one of fluid and tissue, histopathologically assessing the tissue, and measuring at least one of volume and weight of the at least one of fluid and tissue.

20. The method of claim 19, wherein the fluid obtained from at least one of the first and second human kidneys is perfusate, urine, and/or blood.
